(19) United States
(12) Patent Application Publication
(10) Pub. No.: US 2005/0007550 A1
(43) Pub. Date: Jan. 13, 2005

(54) System and method for relieving eye strain

(76) Inventors: Yury Turkov, British Columbia (CA); Elena Anikina, British Columbia (CA); Damien Loveland, British Columbia (CA); Faouzi Zerrouk, Richmond (CA)

Correspondence Address: Hall Priddy;Myers & Vande Sande, 200 10220 River Road, Potomac, MD, 20854, US

(21) Appl. No.: 10/488,761

(22) PCT Filed: Sep. 6, 2002
(86) PCT No.: PCT/CA02/01368

(30) Foreign Application Priority Data
Sep. 6, 2001 (CA) 2357432

(51) Int. Cl. A61B 3/00

(52) U.S. Cl. 351/203

(57) ABSTRACT

The invention provides methods and apparatuses for vision therapy procedures using laser interference patterns for relieving eye strain. One of the methods provided controls a speckle pattern in such a manner that an interesting visual stimulus is created which attracts and retains a viewer's attention and forces the eye to adjust to distance vision. This stimulates relaxation of the ciliary muscles of the viewer's eyes and contributes to the relief of eye strain. Another method provides electronically generated holograms for exercising eye muscles in order to relieve eye strain. The present invention provides further methods and apparatuses which may be used in a computer user's workplace.

### FIELD

The present invention relates to the field of vision therapy. More specifically, the present invention concerns the methods and systems for laser therapy of eye fatigue and eye strain conditions arising from excessive computer use and other near-work.

### BACKGROUND

Recently, a dramatically growing proportion of the population suffers from visual disorders connected with eye fatigue and eye strain due to excessive computer use. The prime cause of these disorders is a prolonged fixation of the eye on near objects during computer work. The computer display screen in particular belongs to this group of objects.

To focus the eye on closely located objects, a contraction of the ciliary muscle is needed. The ciliary muscle controls the accommodation of the eye's lens according to the distance of the objects to be focused upon. The ciliary muscles must contract to adjust for near vision, and must relax to adjust for distance vision. If the ciliary muscle is in a contracted state for a sufficiently long time period, as occurs during prolonged computer use, the biophysical and biochemical processes in its tissues change. An impairment of hemodynamics occurs, bringing about an impaired nutrition of the ciliary muscle and an impairment of metabolism. Changes also include vegetation of the network of nerves of the accommodation apparatus. Similar processes also take place in the muscles controlling eye convergence, i.e. the ability to turn the eyes inward to maintain single vision when viewing close objects.

Eventually, the changes lead to a decrease in both the accommodative capacity of the eye and the ability to converge, bringing on eye strain and development of various visual disorders such as accommodative asthenopia, rapid progression of myopia, etc. (Leonard J. Press, Applied Concepts in Vision Therapy, Mosby, 1997, at p.298). The complex of ocular and visual problems related to computer use has been termed as “Computer Vision Syndrome” or CVS. Most studies show that from 75% to 90% of computer users experience the symptoms of CVS (James E. Sheedy, “Should you provide Eye Care for your Computer Workers”, http://www.cvsdoctors.com/eyecare.doc, 1999).

At the present, various methods for prophylaxis and the relief of eye strain connected with excessive computer use are known. Among these methods, the ones of greatest interest are those which can be realized in the computer user's workplace. Apart from advantages related with the possibility of using the computer itself for controlling the vision therapy process, the main advantage of such methods is the opportune possibility of their operation and application by the computer user himself, without professional medical assistance.

Widely used methods of relieving eye strain involve the observation of specific, dynamic, computer-controlled images formed on the computer display screen (see U.S. Pat. No. 6,042,231 issued Mar. 28, 2000 to Fateh). The principal drawback of these vision therapy methods is that the images are in fact planar, i.e. two-dimensional. Therefore, regardless of the illusion of 3D, the viewer's eyes must be accommodated on the plane of the display screen for clear vision. Consequently, such computer methods are not of great efficiency for the elimination of visual dysfunctions connected with accommodative eye disorders, especially if the viewer does not apply any additional lenses while using the methods as, for example, described by Press (at p.229).

Among the therapy methods directed at the elimination of accommodative eye disorders and the relief of eye strain, methods using a laser speckle pattern, i.e. a random fine-grained interference pattern, are of great interest. The visual perception of a laser speckle pattern differs considerably from the perception of other physical objects, which are observed in everyday life. The difference is due to the fact that the speckle pattern is always perceived as a clear, high-contrast image independently of the optical condition of the eye (ametropia, clouding of the crystalline lens, haziness of vitreous body etc.) and the accommodative state of the eye. The perceived image consists of many randomly located, generally small, grains (speckles) and comprises a wide spectrum of spatial frequencies. The upper bound of the perceived spatial frequency depends on the resolving ability of an individual's visual system, from retina to brain cortex, whereas the lower bound depends on the size of the features on the scattering object. The most important feature of a laser speckle pattern, that it is perceived as a clear image independently of the distance the eye is focused on, allows the process of accommodation to be eliminated from the act of vision. Due to the above features, the laser speckle pattern has ophthalmic applications for both diagnostics and therapy of the eye's optical and sensory apparatus.

In some ophthalmic applications designed for diagnostic and therapeutic purposes, moving speckle patterns are used. In these cases the movement is usually a simple translation of the whole speckle pattern. Such moving speckle patterns can be created, for example, by means of laser light scattering from a moving diffuse surface (as in U.S. Pat. No. 3,724,933 issued Apr. 3, 1973 to Mohon et al.; U.K. Patent 2,205,661A issued December 1988 to Ley) or by means of scanned laser light scattering from a stationary diffuse screen (as in Russian Patent No. 5,566,471/14, issued July 1995 to Zavalishin et al.). In both cases, the observer perceives a moving speckle image. The direction and the speed of speckle motion perceived by the observer depend on the eye's refractive conditions. In the cases of myopic (nearsightedness) and hyperopic (farsightedness) eyes, the directions of motion are opposite. The speed of the speckle depends on the severity of the eye's condition. Electronic control of the motion of a speckle pattern is also possible. One technical solution using liquid crystals is an eye exercising option for the device with mechanically moving features described by Ley.

Disadvantages of the prior art for relaxing the eyes include the use of mechanically moving parts, the absence of any interesting stimulus for the observer, the unsuitability of the inventions to the computer user's workplace, and the need for a medical assistant. However, the main disadvantage of the prior art is that it does not use specific means for purposefully forcing the eye into accommodation for distance vision, which is necessary to relieve eye strain.

Further methods in the prior art relate to the relief of eye strain through the exercising of the ciliary and converging muscles of the eyes. The most effective method of accommodative eye exercising consists in alternate observation of various objects located at different distances from the viewer's eyes, or observation of a movable object located at variable distances.

In particular, computer equipment is known comprising spaced apart displays for showing sequences of images to a viewer (U.S. Pat. No. 4,294,522 issued Oct. 13, 1981 to Jacobs). In one embodiment of this equipment, two displays are disposed, in line, one near to and other distant from the viewer. Each display shows, on a random basis, a series of different images which alternate with the series of images shown by the other display. The viewer alternately observes the near and distant displays, thereby performing an optical exercise. The drawback of this equipment is the necessity of using several (at least, two) computer monitors. This is inconvenient and cost-prohibitive for the application of this method in a workplace.

U.S. Pat. No. 5,173,724 issued Dec. 22, 1992 to Bonham et al., and U.S. Pat. No. 5,040,888 issued Aug. 20, 1991 to Bonham, have suggested a stand-alone optical system for ciliary muscle exercising including a hologram containing multiple images in a single field of view which are located at significantly different distances from the viewer. The choice of images is limited to the selection of holograms.

Yunlong Sheng et al. (JOEL Vol. 9 Supp., Optics for Information Infrastructure, 1998, pp. 84-86) show how holograms can be formed electronically using a spatial light modulator. However, this is unrelated to any application for eye strain relief.

There are some dual monitors in the prior art (U.S. Patent No. RE36,978 reissued Dec. 5, 2000 to Moscovitch, U.S. Pat. No. 5,502,616 issued Mar. 26, 1996 to Maguire, Jr., and U.S. Pat. No. 5,594,620 issued Jan. 14, 1997 to Register) which are intended to increase the available display area compared to a single monitor, however, none of these inventions refer to the use of one screen for regular use and the other for relief of eye strain.

Relating to the current invention and the problem of eye strain relief, greatest emphasis should be put on the methodological aspects of interference pattern control from the point of view of gaining maximum therapeutic effect. Therefore, it is an object of this invention to provide some interesting visual stimulus, which attracts and retains the viewer's attention and forces eye accommodation to realign in such a manner that stimulates the ciliary muscles of the viewer's eyes into relaxing or exercising.

It is another object of this invention to lower the barriers to regular use, in that the invention should be readily available for use as and when needed by a viewer, with minimal effort, and without professional medical assistance.

It is still another object of this invention to provide methods and systems that may be used in new multimedia applications for the purposes of both recreation of the eye and amusement.

### SUMMARY OF THE INVENTION

The invention includes a method for relieving eye strain based upon the observation of a laser interference pattern in the form of a random speckle pattern and/or regular interference pattern and/or three dimensional interference pattern. The displayed interference pattern may be altered dynamically or animated in such a manner that creates an interesting, attention retaining visual stimulus for the viewer. While retaining the viewer's attention, and depending on the type of interference pattern, the stimulus can either lead to relaxing of the ciliary and converging muscles of the viewer's eyes or to their exercise, and therefore contributes to eye strain relief.

The invention further includes an apparatus for realizing the above methods comprising a computer, a laser display unit for displaying a laser interference pattern, which may or may not be integral to the computer, and corresponding computer programs for controlling the laser display and corresponding laser interference patterns. The laser interference display may comprise one or more diffusing light scatterers if a high resolution speckle display is needed, and one or more spatial light modulators for controlling the wavefront of the laser radiation.

In one embodiment the present invention may be a binocular laser speckle display for eye strain relief having two optical channels each displaying a speckle pattern for the viewer's eyes.

Other elements of the present invention include computer programs for controlling the laser display and laser interference patterns, which produce an animated or changing interference pattern in such a manner that stimulates different regimes of accommodation of the viewer's eyes. The computer program may also control a relaxing speckle or holomovie, which will contribute to involuntary eye strain relief while providing interest to the user, and may additionally provide an audible output.

### BRIEF DESCRIPTION OF THE DRAWINGS

The invention itself both as to organization and method of operation, as well as additional objects and advantages thereof, will become readily apparent from the following detailed description when read in connection with the accompanying drawings:

FIG. 1 is a perspective view of a display comprising both a non-laser display unit for displaying alphanumeric, graphical, and other visual information and a laser display unit for relieving eye strain;

FIG. 2 is a diagrammatic view illustrating an optical scheme of a single-color laser speckle display unit;

FIG. 3 is a diagrammatic view illustrating an optical scheme of a two-color laser speckle display unit;

FIG. 4a is a diagrammatic view illustrating optical schemes of laser speckle display units utilizing a single diffusing light scatterer;

FIG. 4b is a diagrammatic view illustrating optical schemes of laser speckle display units, utilizing paired scatterers;

FIG. 4c is a diagrammatic view illustrating the optical scheme of laser speckle display units utilizing dual speckle pattern control;

FIG. 5a is a diagrammatic view illustrating schemes of binocular laser speckle displays for relieving eye strain utilizing a mechanical means for adjusting the space between the axes of the optical channels;

FIG. 5b is a diagrammatic view illustrating schemes of binocular laser speckle displays for relieving eye strain utilizing an electronic means employing liquid crystal spatial amplitude light modulators for adjusting the space between the axes of the optical channels;

FIG. 6 is an illustration of examples of different types of interference images which may be displayed by means of a laser display unit;

FIG. 7 is a diagrammatic view illustrating an optical scheme of a laser display unit;

FIG. 8 is a perspective view of a computer monitor having an added laser display unit.

### DETAILED DESCRIPTION

In accordance with the purpose of the present invention, there are first described the various aspects and aims of the methods and systems for relieving eye strain, followed by a description of a number of specific embodiments.

Method

The first eye strain relief method is implemented using a computer system, with an additional laser display unit for the creation of a speckle pattern, and corresponding software (a computer program) for control of this speckle pattern. The method comprises three steps.

First, a laser speckle pattern is displayed by means of the laser display. Second, an acknowledgement (input) is received indicating that viewer sees the speckle pattern and has identified the features of his perception of the speckle pattern. For instance, the viewer can do this by means of a keyboard or a mouse, and can also indicate the direction of perceived speckle motion. Processing this input allows, in particular, for the recognition of refractive error in the viewer's eyes (e.g. myopia, hyperopia, or astigmatism). Such processing may be introduced as an option to be executed by the viewer on an occasional basis or after a sufficient time period (typically several months) during which the refractive condition of the eye has remained stable.

Third, the displayed speckle pattern is altered dynamically in such a manner that it creates an interesting visual stimulus that causes the viewers eyes to accommodate to distance vision. This visual stimulus is formed after taking into account the input received from the viewer about the viewer's individual perception of the speckle pattern. The observation of such a speckle pattern stimulates the relaxing of the ciliary muscles of the viewer's eyes and contributes to eye strain relief. A visual stimulus could be the illusion of speckles moving away, which could be formed, in particular, by means of suitable dynamic variation of the speckle pattern.

In one related aspect, the eye strain relief method further comprises an additional step consisting of an individual adjusting the speckle pattern parameters. In particular, this adjustment may effect variation of such characteristics as speckle pattern configuration, speckle size, perceived speed of speckle motion, etc. The viewer can carry out the adjustment, as an option, in accordance with the refractive conditions of his eye and his peculiarity of perception of the speckle pattern. The resulting digital data stored in computer memory may be used automatically next time the speckle pattern is needed.

In another related aspect, the eye strain relief method comprises the additional step of transforming the laser speckle pattern in such a manner that it provides visual stimuli which allow testing of the viewer's visual functions.

Another aspect of the present invention is a method for exercising the ciliary muscles of the eyes. The method for exercising the ciliary muscles is implemented using a computer system, an additional laser display for creation of a laser interference pattern, and software for controlling the laser interference pattern. The laser interference pattern is in the form of a three-dimensional image.

The method for exercising the ciliary muscles comprises three steps. First, a three-dimensional interference pattern is displayed by means of the laser display. Second, an input is received indicating that the viewer sees the three-dimensional interference image. Third, the displayed image is dynamically varied such that it creates an interesting visual stimulus, which periodically alternates in a manner that it requires the viewer's eye to adjust between distant and near vision. This technique exercises the ciliary muscles and, as a result, helps to eliminate eye accommodative disorders.

In the most complete implementation of the method of the present invention, a visual therapeutic method is used which is a combination of both the eye strain relief method and the method for exercising the ciliary muscles of the eyes. The visual therapeutic method is implemented using computer system, a laser display for creation of a laser interference pattern, and software for controlling the laser interference pattern. The laser interference pattern may be in the form of a random speckle pattern and/or in form of a regular interference pattern, and/or in form of a three-dimensional image.

The visual therapeutic method comprises three steps. First, a laser speckle pattern is displayed by means of the laser display. Second, an acknowledgement (input) is received indicating that the viewer sees the speckle pattern and has identified the features of the speckle pattern. Third, the displayed speckle pattern is dynamically varied such that it creates an interesting visual stimulus, which periodically alternates in a manner that it requires the viewer's eye to adjust between distant and near vision. During the third step of the above method, the interference pattern may be transformed from a speckle pattern into a regular interference patteror a three-dimensional image. This technique exercises the ciliary muscles and, as a result, helps to eliminate eye accommodative disorders connected, for instance, to eye strain.

It should be especially noted that exercising the ciliary muscles demands a phase of contraction of these muscles. The contraction must alternate with the relaxation phase, which is realized by means of the eye adjusting between near and distant vision.

Apparatus

The methods of the present invention are also embodied in an apparatus for relieving eye strain. The apparatus comprises a computer system, which may be the personal computer of the viewer, a laser speckle display unit coupled with the computer system, and an eye strain relieving application implemented on the computer system.

The laser speckle display unit is designed for speckle pattern displaying. It may be a single-color or multi-color laser display comprising one or more diffusing light scatterers (for example, a ground glass screen) used to create a random interference pattern in the form of a speckle pattern. Control of the resulting speckle pattern can be achieved in a number of ways, two of which are described below.

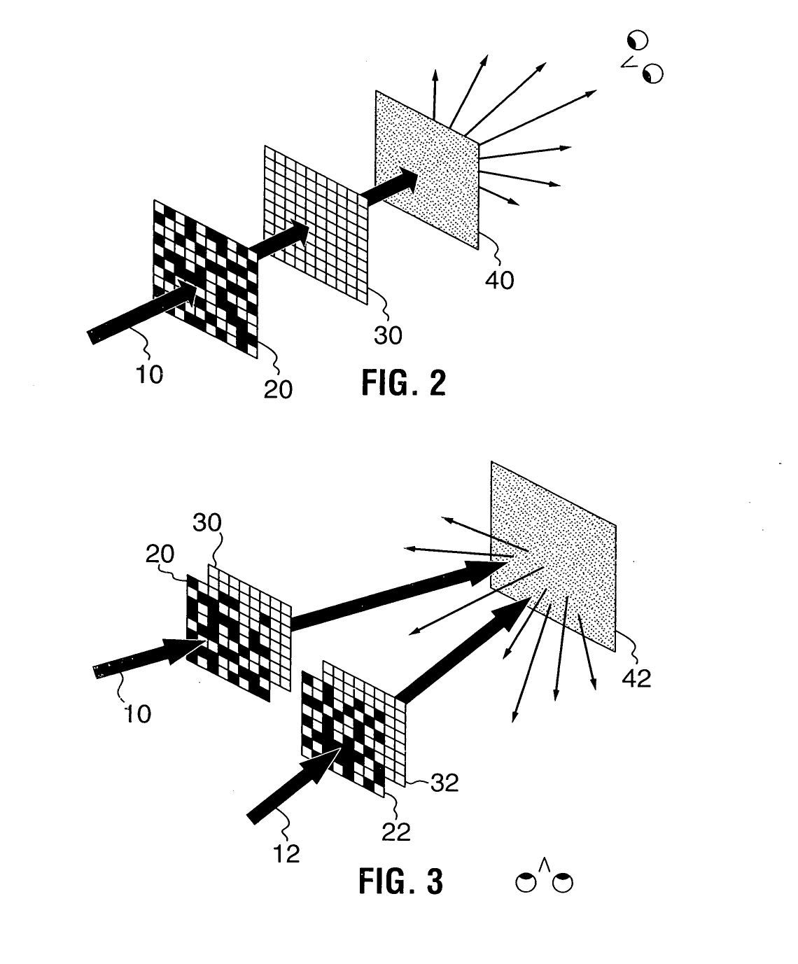

The first way of controlling the speckle pattern is by means of spatial phase and/or spatial amplitude modulation of the laser beams incident upon the diffusing light scatterers. The laser beams may be either expanded or non-expanded. FIG. 2 illustrates the embodiment of single-color laser speckle display unit, which utilizes spatial modulation of the laser beam 10 incident upon the diffusing light scatterer (ground glass screen) 40 in order to realize dynamic control of the speckle pattern in the transmission mode of operation.

The spatial light modulation is achieved by using an amplitude spatial light modulator (SLM) 20 and a phase SLM 30. A viewer observes a laser speckle pattern formed as a result of interference of light beams scattered by the ground glass screen 40. This speckle pattern changes dynamically in accordance with a specified regime of spatial modulation. In particular, an interesting dynamic visual stimulus may be formed, which holds the viewer's attention and stimulates the viewer's eyes to accommodate for distance vision, which in turn contributes to eye strain relief.

FIG. 3 illustrates a laser speckle display unit for the reflection mode of operation (two-color variant). Laser beams 10 and 12, corresponding to different spectral ranges, are spatially modulated by amplitude SLM's 20 and 22 and phase SLM's 30 and 32. A viewer observes a laser speckle pattern formed as a result of interference of light beams back scattered by the ground glass screen 42. It should be noted that, beside the SLM's, the laser speckle display unit may comprise some additional optical elements (e.g. lenses, mirrors, etc.) for manipulation of the laser beams. This is true for all laser speckle display unit embodiments described in this specification.

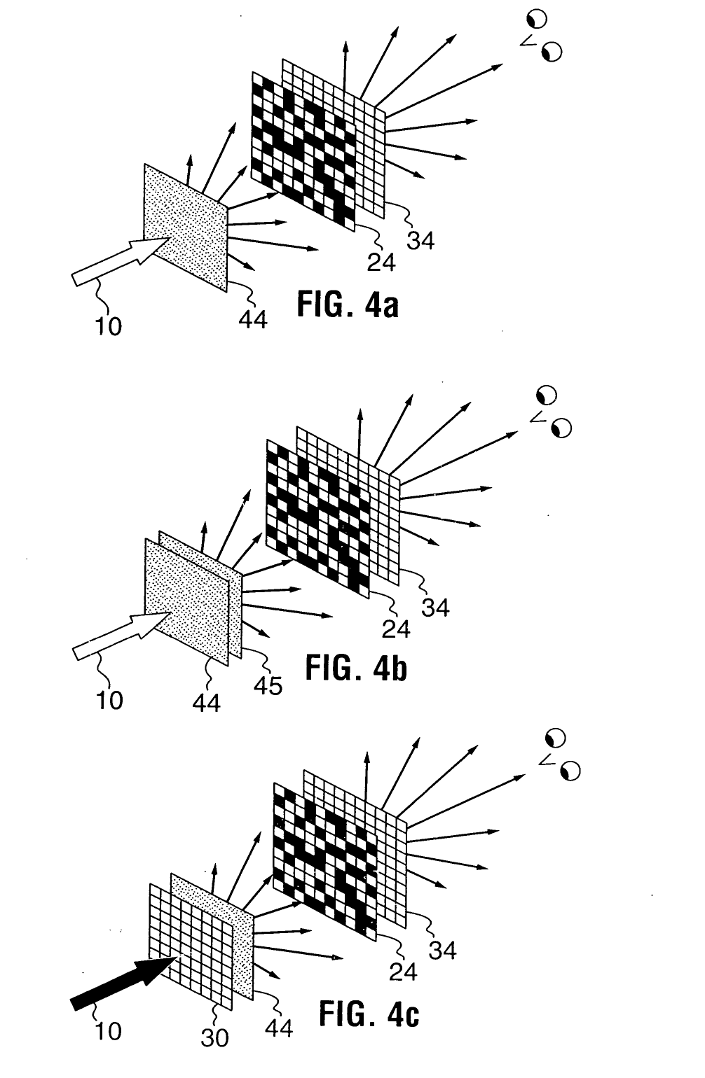

The second way of controlling the speckle pattern is by means of spatial modulation of the laser radiation scattered by diffusing light scatterers. This gives more control over the spatial frequency spectrum of the speckle pattern. In particular, it allows for the enlargement of the size of the speckle grains. FIGS. 4a and 4b show possible layouts of the laser speckle display unit, which uses the transmission mode of operation. In FIG. 4a the laser beam 10 falls upon a ground glass screen 44. The scattered beams are spatially modulated by amplitude SLM 24 and phase SLM 34. This spatial modulation affects the wavefront of transmitted laser radiation resulting in a changing speckle pattern, which is observed by the viewer. The arrangement shown in FIG. 4b contains an additional ground glass screen 45, which allows a more uniform spatial distribution of the speckle pattern than in the case of a single diffusing scatterer.

Both of the ways of controlling speckle patterns described above may be combined in a single laser speckle display unit. For example, FIG. 4c shows an arrangement containing an amplitude SLM 24 and a phase SLM 34 which modulate the laser radiation scattered by diffusing light scatterer 44 and a phase SLM 30 which modulates the laser beam 10 incident upon that light scatterer. The benefit of this arrangement is an additional degree of freedom in functional control of the speckle pattern. For example, the phase SLM 30 may be used for setting the dynamic behavior of the speckle pattern while the phase SLM 34 is used for control of the speckle sizes. Both the above SLM's 30 and 34 may operate on different time scales to simplify the software for speckle pattern control.

The laser speckle display unit is preferably implemented as a freestanding apparatus positioned at any place chosen by the viewer. Alternatively, it may be an attachment unit coupled to the computer terminal monitor, keyboard or wall, for example.

The eye strain relief apparatus is controlled by application software implemented on the computer system. The software is responsible for the following functions:
(1) activating and deactivating the laser speckle display either at a time predicted by the computer program or at any moment chosen by the viewer;
(2) receiving and processing acknowledgements indicating that the viewer sees the speckle pattern and has identified the perceived nature of the speckle pattern; and
(3) driving the spatial light modulators for dynamic control of the speckle pattern in such a manner that relaxation of the ciliary muscles of the viewer's eyes is stimulated by encouraging realignment of accommodation to distance vision. The computer program can be activated by the user during a desired rest period, or set to automatically activate at set time intervals, in a similar fashion to, or replacing, a screen saver program.

The application software may further adjust both the parameters and the dynamic characteristics of the speckle pattern in accordance with an individual viewer's demands, as required by the refractive conditions of his eyes and his perception of the speckle pattern. This adjustment may be introduced as an option. Digital data concerning particular settings may be stored in computer memory and used automatically at the following session of the computer program.

The application software may also include a set of digital data used for dynamic control of the laser speckle pattern. The speckle pattern may have different, static shapes, which are changed every few seconds or so, like a slide show. The computer program may combine these patterns into a relaxing speckle movie. Alternatively, the computer program may display an alternating sequence of speckle patterns which is perceived as a moving speckle image. For example, it can create an illusion of streams, flames, rotating spirals, etc. This moving speckle image is designed to be capable of attracting and retaining the viewer's attention. It may be defined as a speckle-clip, which may be accompanied by music. The observation of such speckle-clip will contribute, in particular, to the relief of eye strain.

Alternatively, the laser speckle display can display an interference pattern in the form of a three-dimensional image. It may be a single-color or multi-color laser display comprising one or more phase and/or amplitude SLM's, which are used for creating and controlling the interference pattern. The spatial light modulation may be performed on the basis of liquid crystal or other physical principles.

With the exception of using a three-dimensional image in place of a speckle pattern, the alternative laser display unit is substantially the same as the laser display unit using a speckle pattern as described above.

An additional function of the application software may consist in adjusting the interference pattern parameters and it's dynamic characteristics in accordance with individual viewer's tastes or interests. Furthermore, the application software may include a program to allow testing of the viewer's visual functions. This program provides for three-dimensional displays of test objects or symbols located at different distances from the laser display screen and for processing the viewer's response indicating whether or not he perceives these objects or symbols clearly. For instance, text messages, company logos, advertising slogans, and/or images may be shown.

Another potential use for the application software is the creation of a simple eye exercising holographic movie. The application software would then include computer executable instructions for displaying an alternating sequence of holograms which the viewer perceives as a moving three-dimensional image. For example, the holograms may comprise various geometric figures or other volumetric graphical forms changing their configuration, sizes, orientation, and, most importantly, their distance from the viewer's eyes. This moving hologram image is intended to be capable of attracting and retaining the viewer's sight. It is defined as a holo-clip, which may be accompanied by music. The observation of such a holo-clip will contribute, in particular, to eye exercising and therefore eye strain relief.

Another embodiment of the present invention is a combination system, which can be used both for relief of eye strain and for eye muscle exercising. The system combines the speckle apparatus and the hologram apparatus described above. However, it should be noted that this combination system has limited laser speckle pattern display characteristics due to the limitations of the laser display unit capable of displaying laser speckle patterns and three-dimensional images, as is described below.

Laser Display Unit

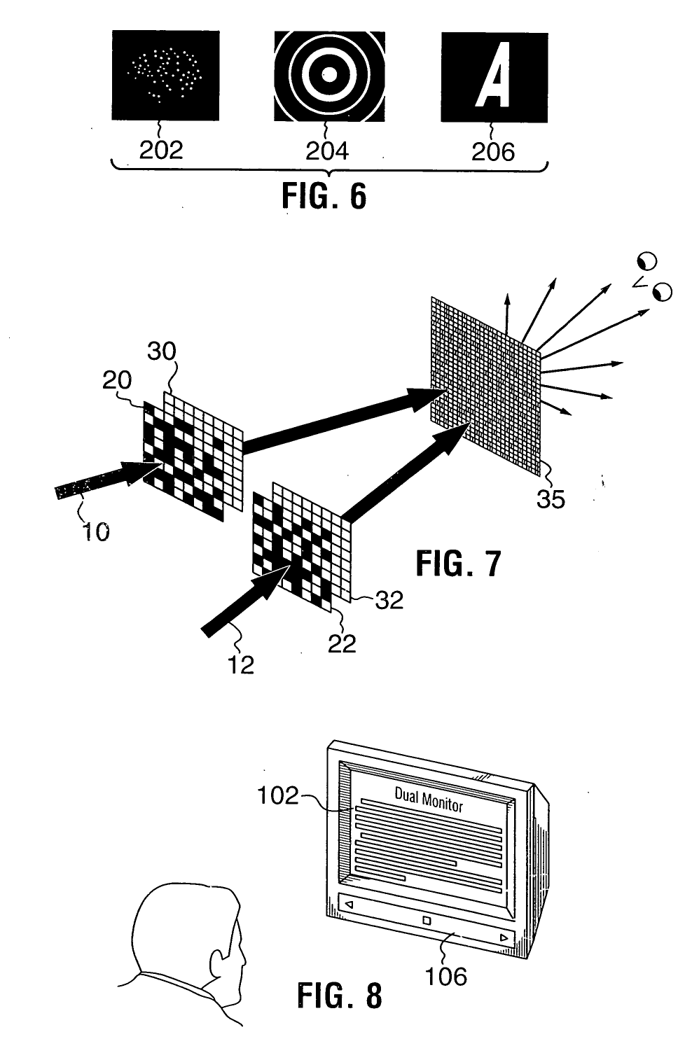

The laser display unit is designed for displaying an interference pattern, which may be in the form of a random speckle pattern, and/or in the form of a regular interference pattern and/or in the form of a three-dimensional image, or any sequential combination thereof. This is illustrated in FIG. 6 where image 202 is an example of speckle pattern displaying, image 204 is a regular interference pattern in the form of concentric circles, and image 206 is a three-dimensional image. The universal laser display may be a single-color or multi-color one. It comprises one or more phase and/or amplitude SLMs, which are used for creating and controlling the interference pattern. The SLMs may be formed on the basis of liquid crystal or other physical principles.

This laser display unit does not comprise any diffusing light scatterers for creating the random interference pattern in the form of a speckle pattern. An SLM having a random phase or amplitude spatial distribution set in accordance with a computer program plays the role of a diffusing scatterer. Although this random light scattering structure is not able to provide spatial frequencies as high as a ground glass screen, it allows the transformation of an interference pattern in a freer manner, including the creation of a random interference pattern, a regular interference pattern or a 3D image.

As an example, FIG. 7 shows a possible arrangement of a two-color laser display unit comprising two paired sets of SLM's including amplitude SLM's 20 and 22 and phase SLM's 30 and 32 which realize a preliminary smooth spatial modulation of two laser beams 10 and 12 which correspond to different spectral ranges. In general, an arbitrary amplitude and phase distribution may be set independently for each laser 10 and 12. A phase SLM 35 introduces small-scale phase inhomogeneities producing a diffraction structure for forming a desirable interference pattern. This interference pattern is observed by a viewer in the transmission mode. If the small-scale phase inhomogeneities induced by SLM 35 are random, the interference pattern will be in form of a speckle pattern.

A dynamic variation of this speckle pattern may be realized using SLM's 20, 22, 30, and 32 or by means of a smooth deformation of the phase diffraction structure performed by SLM 35. The first of these ways is preferable because it allows separate control for two spectral ranges.

Various other embodiments of the laser display units used in systems for eye strain relief and eye exercise are also possible. For example, all the above embodiments of the laser display units may be constructed such that the diffusing light scatterers (ground glass screens) are replaced by phase SLM's.

The laser display unit may be controlled by a computer program in a similar way to the previous embodiments.

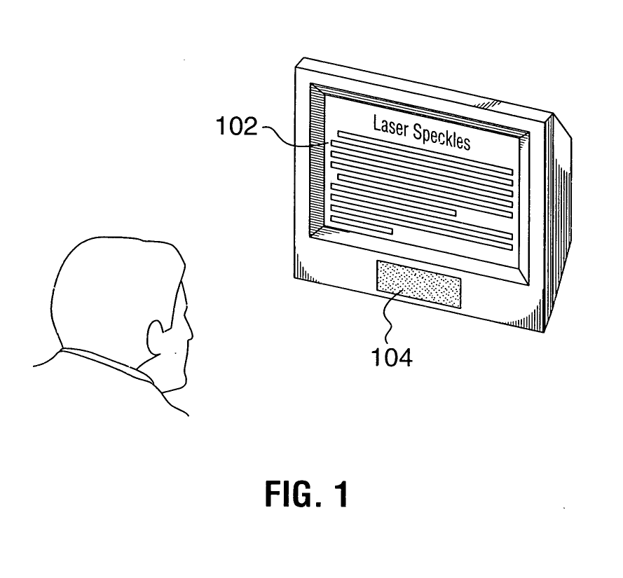

While the laser display unit may be a self-contained unit, it may be preferable to incorporate it directly into an existing display for specific applications. For example, a laser speckle display unit may be incorporated into a basic computer monitor, adding to it a performance capability of relieving the eye strain accumulated in a computer operator's eyes during computer use. As shown in FIG. 1, the computer display comprises both a non-laser visual display unit 102 for alphanumeric, graphical, and other visual information display and a single-color or multi-color laser speckle display unit 104 producing a laser interference pattern for relieving eye strain. The laser speckle display unit comprises one or more diffusing light scatterers for producing a speckle pattern, which contributes to eye strain relief when a computer operator focuses on it. The non-laser display unit can be, for example, an electron-beam tube or a liquid crystal screen. The laser speckle display unit comprises one or more phase and/or amplitude SLMs, which may be formed on the basis of liquid crystal or other physical principles.

An alternative embodiment of the computer monitor incorporating a laser display unit is shown in FIG. 8. The computer monitor includes both a non-laser visual display unit 102 for displaying alphanumeric and graphical information and a single-color or multi-color laser display unit 106 producing a laser interference pattern for eye strain relief and eye exercise. The non-laser display unit can be, for example, an electron-beam tube or a liquid crystal screen. The laser display unit comprises one or more phase or/and amplitude SLMs, which may be formed on the basis of liquid crystal or other physical principles. The interference pattern displayed by the laser display unit may be in an arbitrary form in the form of a three-dimensional image, or a speckle pattern.

The laser display unit has the capability of operating in two different modes:

(a) a dynamic mode. In this mode, a dynamic or changing interference pattern is displayed. The viewer can view this interference pattern at a rest time providing eye exercise depending on the formed visual stimulus; and

(b) a static mode. In this mode, a static three-dimensional interference image is displayed which can consist, for example, of a row of symbols located at different distances behind the display screen. In order to perceive these symbols, the viewer must accommodate the eyes to the corresponding distance. As an option, the symbols may be arranged as computer icons, in which case, a computer operator would perceive them and click them periodically in accordance with performing an unrelated computer task. In this manner, for example, the saving of a text document may be connected with the necessity of eye fixing into distance.

The computer monitor may have a non-laser visual display unit and a laser display unit, both using the same liquid crystal panel. The liquid crystal panel may be divided into two separate functional parts. One of these functional parts is the regular, non-laser visual information display unit of the monitor, and other of these functional parts is used by the laser display unit.

Another embodiment is for the computer-based laser speckle display unit to be controlled by computer game software. In this case, the software could be set to automatically and periodically switch off the standard non-laser display unit and switch on the laser-speckle display unit for the purpose of regular eye relaxing. The game could continue using an animated laser speckle display. This automation would eliminate the problem of a child's reluctance to break away from a computer screen, and overcome the difficulty associated with a child's normal unawareness of the necessity to look after his or her eyes.

Portable or dedicated video game machines may incorporate the same feature. Also, a laser speckle display unit could be incorporated into other electronic equipment where its use would be opportune, convenient and take minimal effort on behalf of the user, such as in a telephone, TV set or personal digital assistant. For instance, someone may observe, as an involuntary reflex, a speckle pattern when he or she speaks on the telephone during a break in computer work, thereby relieving eye strain. Likewise, a TV viewer may relieve eye strain as opportunity offers. Even some advertising clips may be connected with speckle pattern observation.

Binocular

Another embodiment of the present invention is a binocular laser speckle display. The binocular display has two optical channels each displaying a speckle pattern for each of the viewer's eyes. A viewer, without any binocular dysfunction, perceives the speckle patterns observed by two eyes as a single speckle image when the space between the axes of the optical channels is adjusted in accordance with his individual interpupillary distance. Therefore, the binocular laser speckle display includes a means for such adjustment. The adjustment means can be a mechanical module or an electronic assembly using, for example, liquid crystal amplitude SLM's.

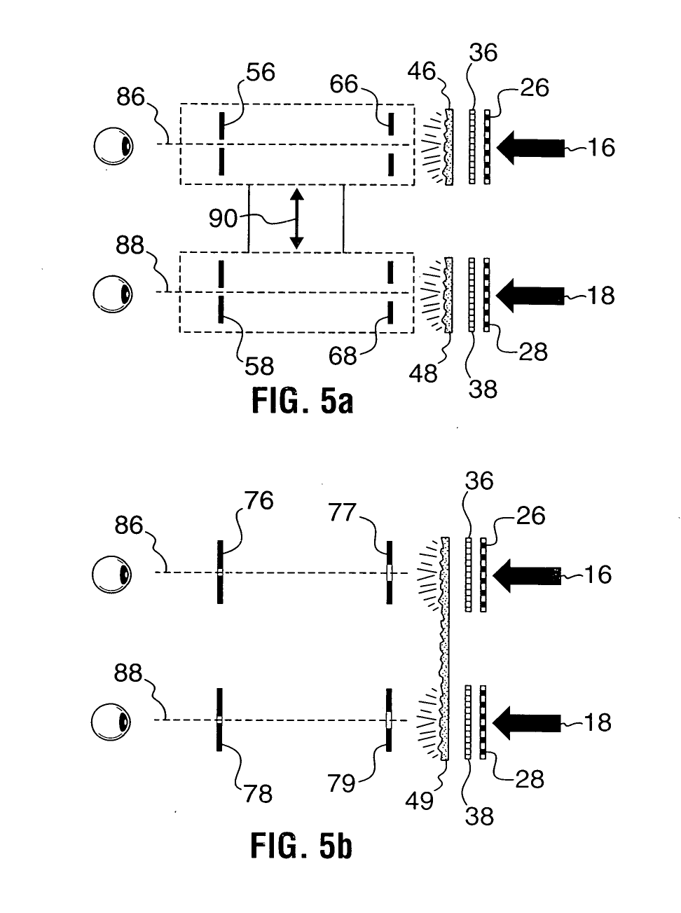

FIG. 5a is a schematic drawing of a binocular laser speckle display having a mechanical module 90 for adjusting the spacing between the optical channels. Laser beams 16 and 18 (in left and right channels, respectively), are spatially modulated by amplitude SLM's 26 and 28 and phase SLM's 36 and 38, and illuminate ground glass screens 46 and 48. A viewer observes the resulting speckle patterns with two eyes in the directions 86 and 88, which are the optical axes of the two channels. The optical axes are set by input apertures 66 and 68 and output apertures 56 and 58. These apertures affect to a certain degree the structure of perceived speckle pattern.

FIG. 5b is a schematic drawing of a binocular laser speckle display having an electronic assembly for adjusting the spacing between the optical channels. The electronic assembly comprises four liquid crystal amplitude SLM's: 76 and 77 for the left optical channel; 78 and 79 for the right channel. Each SLM is controlled in such a manner that a transparent window is formed. The windows formed by SLM's 76 and 78 are output apertures of optical channels, and the windows formed by SLM's 77 and 79 are input apertures. The positions of these apertures define the axes of the optical channels, which determine the directions 86 and 88 of perceived speckle pattern movement. In this example, a single diffusing light scatterer 49 is utilized for forming speckle patterns in both optical channels. Control of the speckle pattern is performed by amplitude SLM's 26 and 28 and phase SLM's 36 and 38, as in the above example shown in FIG. 5a. Advantages of a binocular laser speckle display include using small SLMs for controlling the speckle patterns, making the device more portable than a device incorporated into a computer monitor.

Advantageously, an alternative embodiment of the binocular laser speckle display is able to produce both speckle patterns and regular interference patterns for each optical channel. This type of binocular laser display may be realized, for example, by replacing the diffusing light scatterers 46, 48, and 49 by corresponding phase SLM's.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the scope of the invention.

1. A method for relieving eye strain, eliminating eye accommodative disorders, and exercising the ciliary muscles of the eyes, comprising:
(a) activating a laser display which displays a laser interference pattern in the form of a random speckle pattern;
(b) receiving an input from a viewer indicating that said viewer perceives said laser interference pattern; and
(c) producing a dynamic alteration of said laser interference pattern;
wherein said dynamic alteration of said laser interference pattern is operative, by means of producing visual stimuli, to stimulate changing of eye accommodation of said viewer.

2. The method according to claim 1, wherein said changing of eye accommodation comprises realignment of said eye accommodation for distance vision, operative to stimulate ciliary muscles of said viewer into relaxing.

3. The method according to claim 1, wherein said changing of eye accommodation comprises realignment of said eye accommodation periodically for distant and near vision in turn, operative to stimulate ciliary muscles of said viewer into exercising.

4. The method according to claim 1, wherein said dynamic alteration of said laser interference pattern involves its transformation into the form of a three-dimensional image.

5. The method according to claim 1, wherein said dynamic alteration of said laser interference pattern involves its transformation into a regular interference pattern.

6. The method according to claim 1, including the additional step of adjusting the display parameters of said laser interference pattern in response to said input from said viewer.

7. The method according to claim 1, wherein said dynamic alteration of said laser interference pattern can be controlled to allow testing of visual functions of said viewer.

8. The method according to claim 1, wherein said dynamic alteration of said laser interference pattern is controlled by a computer software program.

9. The method according to claim 1, wherein said laser display is coupled to a computer monitor.

10. The method according to claim 1, wherein said laser display is a single-color laser display.

11. The method according to claim 1, wherein said laser display is a multi-color laser display.

12. The method according to claim 1, wherein said laser display is automatically activated by a computer software program.

13. The method according to claim 12, wherein said computer software program is a screen saver.

14. The method according to claim 12, wherein said computer software program is a video game.

15. An apparatus for relieving eye strain and for exercising eye muscles, comprising:
(a) a computer;
(b) a laser interference pattern display unit coupled to said computer, comprising one or more spatial light modulators, and one or more diffuse light scatterers, and
(c) a computer software program implemented on said computer operative to control said laser interference pattern display unit;
wherein said laser interference pattern display unit produces a laser interference pattern which is dynamically varied so as to require a viewer's eye to adjust between distant and near vision in accordance with instructions of said computer software program and in order to relax or, alternatively, relax and contract ciliary muscles of said viewer's eye.

16. The apparatus according to claim 15, wherein said laser interference pattern display unit produces a speckle pattern that can be changed in response to input from a viewer as to his perception of the speckle pattern.

17. The apparatus according to claim 15, wherein said laser inference pattern display unit is coupled to a computer monitor connected to said computer such that a viewer of said computer monitor can input instructions to said computer which control operation of said laser interference pattern display unit.

18. An apparatus according to claim 15, including one or more laser radiation sources;
wherein said one or more spatial light modulators control phase and/or amplitude characteristics of laser radiation generated by said one or more laser radiation sources, said laser radiation being either incident upon or transmitted through said one or more diffusing light scatterers, to provide said laser interference pattern display.

19. The laser speckle display unit according to claim 18, wherein said speckle pattern is formed by interference of laser light transmitted through said one or more diffusing light scatterers.

20. The laser speckle display unit according to claim 18, designed in a binocular format, comprising:
(a) two optical channels, each displaying a dynamically alterable speckle pattern for one eye;
(b) a means for adjusting the optical channels in accordance with the distance between the eyes of said viewer.

21. The laser speckle display unit according to claim 20, wherein said means for adjusting the optical channels is an electronic assembly comprising one or more liquid crystal spatial light modulators forming electronically controlled output and input apertures of said optical channels.

22. A computer-readable medium containing a computer program for relieving eye strain, comprising:
(a) computer executable instructions for controlling an eye strain relief device, comprising:
(i) a first instruction for activating and deactivating a laser display operative to display a laser interference pattern;
(ii) a second instruction for processing an input from a viewer indicating that said viewer perceives said laser interference pattern; and
(iii) a third instruction for producing a dynamic alteration of said laser interference pattern to stimulate relaxation of ciliary muscles of said viewer;
(b) a set of digital data for generating a plurality of laser interference patterns; and
(c) a set of digital data for dynamic control of said laser interference patterns.

23. The computer-readable medium according to claim 22, wherein said computer program further includes computer executable instructions for adjusting various display parameters of said laser interference pattern in response to a demand input from said viewer.

24. The computer-readable medium according to claim 22, wherein said computer program further includes computer executable instructions for controlling said laser interference pattern to allow testing of visual functions of said viewer.

25. The computer-readable medium according to claim 22, wherein said computer program further includes computer executable instructions for providing audible feedback to said viewer during operation of said computer program.

26. The computer-readable medium according to claim 25, wherein said computer program further includes a set of digital data for use in proving said audible feedback.

27. The computer-readable medium according to claim 22, wherein said computer program further includes computer executable instructions for arranging a sequence of said laser interference patterns of fixed time intervals in such a way that said laser interference patterns are perceived by said viewer as a continuous moving image, said sequence of laser interference patterns defined as a holo-clip (or a speckle-clip in the case of a speckle pattern display), wherein observation of said holo-clip (or speckle-clip) by said viewer relieves eye strain by stimulating relaxation or exercise of ciliary muscles of said viewer.

28. The computer-readable medium according to claim 22, wherein said laser interference patterns are random speckle patterns.

29. The computer-readable medium according to claim 22, wherein said laser interference patterns are regular interference patterns.

30. The computer-readable medium according to claim 22, wherein said laser interference patterns are three-dimensional images.

31. The computer-readable medium according to claim 22, wherein said laser interference patterns are a combination of random speckle patterns and three-dimensional images.

32. An image display unit capable of providing eye strain relief and eye exercise in addition to displaying conventional alphanumeric, graphic, and other types of images, comprising:
(a) a visual display unit for displaying alphanumeric, graphic, and other visual information, said visual display unit using a display means selected from the group consisting of: an electron-beam tube, a liquid crystal display, and other non-laser display means; and
(b) a laser display unit comprising one or more spatial light modulators, said laser display unit operative to display laser interference patterns, said laser interference patterns selected from the group consisting of: random speckle patterns, regular interference patterns, and three-dimensional images;
wherein said laser interference patterns contribute to the relief of eye strain and exercising of eyes when a viewer looks at said laser display unit.

33. The image display unit according to claim 32, wherein said visual display unit and said laser display unit both use a single liquid crystal panel.

34. The image display unit according to claim 32, wherein said image display unit is a television set.

35. The image display unit according to claim 32, wherein said image display unit is a computer monitor.

36. The image display unit according to claim 32, wherein said image display unit is integrated into a personal digital assistant.

37. The image display unit according to claim 32, wherein said image display unit is integrated into a game-playing device.

38. The image display unit according to claim 32, wherein said image display unit is integrated into a telephone.
